(19) United States
(12) Patent Application Publication
(10) Pub. No.: US 2024/0222891 A1
(43) Pub. Date: Jul. 4, 2024

(54) PCBA PIG TAIL SPRING CONNECTOR

(71) Applicant: Cardiac Pacemakers, Inc., St. Paul, MN (US)

(72) Inventors: James Michael English, Cahir (IE); Moira B. Sweeney, St. Paul, MN (US); Benjamin J. Haasl, Forest Lake, MN (US)

(21) Appl. No.: 18/398,523

(22) Filed: Dec. 28, 2023

Related U.S. Application Data
(60) Provisional application No. 63/435,904, filed on Dec. 29, 2022.

(51) Int. Cl. H01R 12/63; A61N 1/375; H01R 43/02; H05K 1/11

(52) U.S. Cl. CPC H01R 12/63; A61N 1/3754; H01R 43/02; H05K 1/115; H05K 2201/10189; H05K 2201/10356

(57) ABSTRACT

An implantable medical device can include a housing including electronic devices within the housing; a header attached to the housing and including one or more bores; and a feedthrough assembly between the housing and the header; wherein the electronic devices include a PCB electronically connected to the header by a feedthrough wire running from the feedthrough assembly to the PCB, wherein the feedthrough wire is connected to the PCB with a pigtail spring connector.

### TECHNICAL FIELD

This application claims the benefit of U.S. Provisional Application No. 63/435,904, filed on Dec. 29, 2022, which is hereby incorporated by reference in its entirety.

### TECHNICAL FIELD

Various embodiments described herein relate to apparatus, systems, and methods associated with implantable medical devices.

### BACKGROUND

An ambulatory medical device, such as an implantable medical device (IMD), can be configured for implant in a subject, such as a patient. An IMD can be configured to be coupled to a patient's heart such as via one or more implantable leads. Such an IMD can obtain diagnostic information or generate therapy to be provided to the patient, such as via the coupled implantable lead.

In one configuration, IMDs have a header that is coupled to a housing that houses much of the electronics of the IMD. The header can be used to couple a conductor of the lead with circuitry within the implantable device. The header can be electrically connected to the electronics in the housing by one or more feedthrough wires attached to a PCB within the housing.

### SUMMARY

Example 1 can include subject matter such as an implantable medical device. The implantable medical device can include a housing including electronic devices within the housing; a header attached to the housing and including one or more bores; and a feedthrough assembly between the housing and the header; wherein the electronic devices include a PCB electronically connected to the header by a feedthrough wire running from the feedthrough assembly to the PCB, wherein the feedthrough wire is connected to the PCB with a pigtail spring connector.

In Example 2, the subject matter of Example 1 can optionally include the pigtail spring connector including a curled shape.

In Example 3, the subject matter of any one or more of Examples 1-2 can optionally include the pigtail spring connector including a coil shape.

In Example 4, the subject matter of any one or more of Examples 1-3 can optionally include the pigtail spring connector being attached to the PCB at a through-hole trace contact on a surface of the PCB.

In Example 5, the subject matter of any one or more of Examples 1-4 can optionally include the pigtail spring connector including an axis extending perpendicularly to the PCB surface.

In Example 6, the subject matter of any one or more of Examples 1-5 can optionally include the pigtail spring connector including a coil shape having an axis extending perpendicular from a surface of the PCB, wherein the coil shape defines a series of diameters that go from larger to smaller as the coil shape extends farther from the PCB surface.

In Example 7, the subject matter of any one or more of Examples 1-6 can optionally include the pigtail spring connector including a gold-coated wire coil.

In Example 8, the subject matter of any one or more of Examples 1-7 can optionally include the feedthrough wire extending through a central axis of the pigtail spring connector so as to contact an inner surface of a coil shape of the pigtail spring connector.

In Example 9, the subject matter of any one or more of Examples 1-8 can optionally include the feedthrough wire extending through a through-hole in the PCB and going up though the pigtail spring connector and being tack welded to the pigtail spring connector.

In Example 10, the subject matter of any one or more of Examples 1-9 can optionally include the pigtail spring connector including a gold-coated wire coil.

Example 11 can include a method of making a feedthrough wire connection to a PCB for an implantable medical device. The method can include attaching a pigtail spring connector to a PCB; and connecting a feedthrough wire to the pigtail spring connector.

In Example 12, the subject matter of any one or more of Examples 1-11 can optionally include the pigtail spring connector including a coil shape.

In Example 13, the subject matter of any one or more of Examples 1-12 can optionally include the pigtail spring connector being attached to the PCB at a through-hole trace contact on a surface of the PCB.

In Example 14, the subject matter of any one or more of Examples 1-13 can optionally include the pigtail spring connector including an axis extending perpendicularly to the PCB surface, and wherein the coil shape defines a series of diameters that go from larger to smaller as the coil shape extends farther from the PCB surface.

In Example 15, the subject matter of any one or more of Examples 1-14 can optionally include the pigtail spring connector including a gold-coated wire coil.

In Example 16, the subject matter of any one or more of Examples 1-15 can optionally include the feedthrough wire extending through a central axis of the pigtail spring connector so as to contact an inner surface of a coil shape of the pigtail spring connector.

In Example 17, the subject matter of any one or more of Examples 1-16 can optionally include the feedthrough wire extending through a through-hole in the PCB and going up though the pigtail spring connector and being tack welded to the pigtail spring connector.

Example 18 can include subject matter such as a method of attaching a feedthrough wire to a PCB. The method can include extending a feedthrough wire from a feedthrough assembly through a through-hole in a PCB and through a center of a pigtail spring connector; and tack-welding the feedthrough wire to the pigtail spring connector.

In Example 19, the subject matter of any one or more of Examples 1-18 can optionally include the pigtail spring connector being attached to the PCB at a trace contact at the through-hole of the PCB, wherein the pigtail spring connector includes an axis extending perpendicularly to a surface of the PCB, and wherein the pigtail spring connector includes a coil shape having an axis extending perpendicular from the surface of the PCB, wherein the coil shape defines a series of diameters that go from larger to smaller as the coil shape extends farther from the PCB surface.

In Example 20, the subject matter of any one or more of Examples 1-19 can optionally include the feedthrough wire extending through a central axis of the pigtail spring connector so as to contact an inner surface of the pigtail spring connector.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

FIG. 1 shows an example implantable medical device, in accordance with one embodiment.

FIG. 2 shows a side view of a PCB, in accordance with one embodiment.

FIG. 3 shows a top view of the PCB of FIG. 2, in accordance with one embodiment.

FIG. 4 shows a perspective view of a pigtail spring connector, in accordance with one embodiment.

FIG. 5 shows a side view of the PCB, in accordance with one embodiment.

FIG. 6 shows a method of making a feedthrough wire connection to a PCB, in accordance with one embodiment.

FIG. 7 shows a method of attaching a feedthrough wire to a PCB, in accordance with one embodiment.

### DETAILED DESCRIPTION

In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized and structural, logical, and electrical changes may be made.

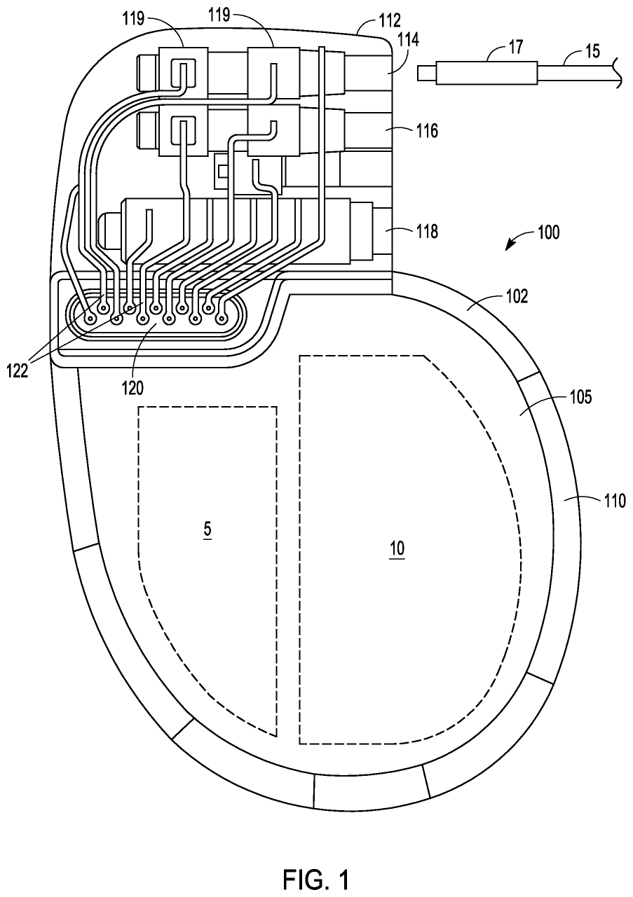

FIG. 1 shows an implantable system 100 including an implantable medical device 102, in accordance with one embodiment. The implantable medical device 102 includes a pulse generator 105 and at least one implantable lead 15. The pulse generator 105 includes a housing 110 and a header 112 mounted to the housing 110. The pulse generator 105 can be implanted into a subcutaneous pocket made in the wall of a patient's chest. Alternatively, the pulse generator 105 can be placed in a subcutaneous pocket made in the abdomen, or in other locations. Pulse generator 105 can include electronic devices such as a power supply 5 including a battery, a capacitor, and other components housed in the housing 110. The pulse generator 105 can further include other electronic devices such as microprocessors 10 to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia.

The header 112 can include one or more bores 114, 116, 118 to receive an implantable lead 15. The implantable lead 15 can include electrodes on a distal end to provide therapy to a body and include a terminal pin 17 on the proximal end to couple to the bore 114, 116, 118. At least one electrical conductor is disposed within the lead 15 and extends from the proximal end to the electrode. The electrical conductor carries electrical currents and signals between the pulse generator 105 and the electrode.

Contacts on the terminal pin 17 can electrically contact electrical contacts 119 within the bores 114, 116, 118 to allow signals and therapy to be delivered to and from electrodes in a body to the electronic devices 5, 10 within the housing 110. The contacts 119 can be connected by wires to a feedthrough assembly 120 to electrically communicate between the lead 15 and the electronic devices 5, 10 within the housing 110.

As noted, the pulse generator 105 includes electronic devices such as microprocessors 10 to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia.

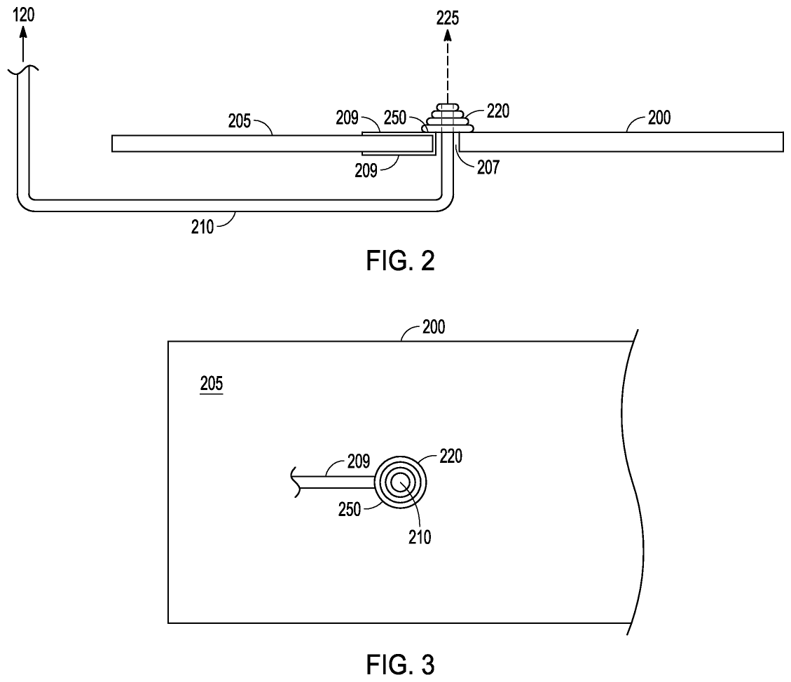

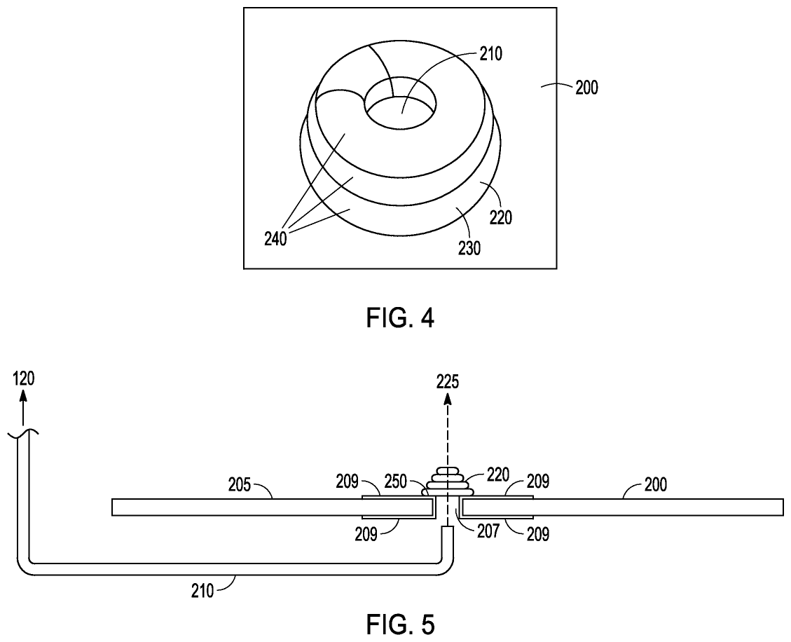

FIG. 2 shows a side view of a printed circuit board assembly (PCB 200) within the housing, in accordance with one embodiment, FIG. 3 shows a top view of the PCB 200, and FIG. 4 shows a perspective view of a pigtail spring connector 220, in accordance with one embodiment.

Here, the electronic devices 10 of the pulse generator 105 (FIG. 1) can include the PCB 200 electronically connected to the header 112 (FIG. 1) by a feedthrough wire 210 running from the feedthrough assembly 120 to the PCB 200.

One challenge is to make the connection to the PCB 200 easier from a manufacturing point of view, and preferably not having to make a solder connection. Accordingly, in this example, the feedthrough wire 210 can be connected to the PCB 200 with a pigtail spring connector 220. In actual use, there can be a plurality of feedthrough wires 210 running between the feedthrough assembly 120 and the PCB 200. However, for ease of description only the single feedthrough wire 210 will be discussed.

In general, the pigtail spring connector 220 includes a curled shape. More specifically, the pigtail spring connector 220 includes a coil shape. For example, the pigtail spring connector 220 can include a gold-coated wire 230 wound in a joined sequence of concentric coils 240 of different diameters defining a coiled spring.

In one embodiment, the pigtail spring connector 220 can be attached to the PCB 200 at a through-hole trace contact 250 on a surface 205 of the PCB 200. The PCB 200 can include a plurality of through holes 207 with contacts 250 leading to one or more traces 209 that can run along the top and/or bottom surfaces of the PCB 200.

The pigtail spring connector 220 can include a central axis 225 extending perpendicularly to the PCB surface 205, and the coil shape can define a series of diameters that go from larger to smaller as the coil shape extends farther from the PCB surface 205.

The feedthrough wire 210 extends through the central axis 225 of the pigtail spring connector 220 so as to contact an inner surface of a coil shape of the pigtail spring connector 220. The diminishing shape of the coils 240 as the feedthrough wire 210 enters the coils of the pigtail spring connector 220 grips the feedthrough wire and holds the feedthrough wire 210 in place relative to the PCB

Referring also to FIG. 5, which shows side view of the PCB 200 before the feedthrough wire 210 enters the pigtail spring connector 220, the feedthrough wire 210 can be extended through the through-hole 207 in the PCB 200 and up though the axis 225 of the pigtail spring connector 220 and can then be tack welded to the pigtail spring connector 220. Such an assembly technique allows the manufacturer to make a connection to the PCB assembly without soldering.

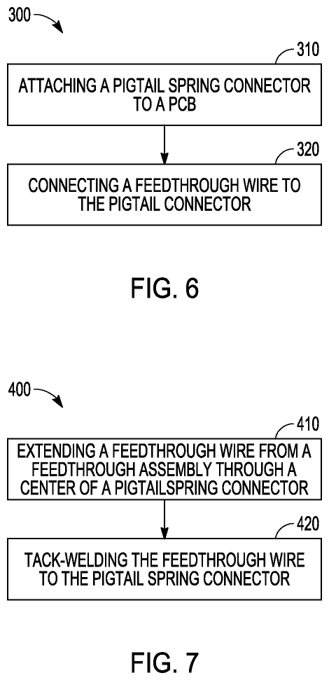

FIG. 6 shows a method (300) of making a feedthrough wire connection to a PCB, in accordance with one embodiment. Referring also to FIGS. 2-5, the method (300) can include attaching a pigtail spring connector 220 to a PCB (310); and connecting a feedthrough wire 210 to the pigtail spring connector 220 (320).

In one embodiment, the pigtail spring connector can be pre-attached to the PCB 200 at the trace contact 250 by welding, soldering, or other attachment techniques before the final assembly of the implantable medical device 102. As discussed, the pigtail spring connector can include a coil shape, and be attached to the PCB at the through-hole trace contact 250 on a surface of the PCB 200.

Again, the pigtail spring connector 220 can include an axis extending perpendicularly to the PCB surface, and the coil shape can have a diameter that goes from larger to smaller as the coil shape extends farther from the PCB surface. The feedthrough wire 210 can be inserted through the central axis of the pigtail spring connector so as to contact an inner surface of a coil shape of the pigtail spring connector. Then, the feedthrough wire 210 can be connected by being tack-welded to the pigtail spring connector 220. Again, the present system provides a simple connecting technique which does not require soldering.

FIG. 7 shows a method (400) of attaching a feedthrough wire to a PCB, in accordance with one embodiment. The method (400) can include extending a feedthrough wire from a feedthrough assembly (410) through a through-hole in a PCB and through a center of a pigtail spring connector; and tack-welding the feedthrough wire to the pigtail spring connector (420).

Again, the pigtail spring connector can be attached to the PCB at a trace contact at a through-hole of the PCB. The pigtail spring connector can include an axis extending perpendicularly to a surface of the PCB, and the pigtail spring connector can include a coil shape having an axis extending perpendicular from the surface of the PCB. The coil shape can have a diameter that goes from larger to smaller as the coil shape extends farther from the PCB surface.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable medical device comprising:
a housing including electronic devices within the housing;
a header attached to the housing and including one or more bores; and
a feedthrough assembly between the housing and the header;
wherein the electronic devices include a PCB electronically connected to the header by a feedthrough wire running from the feedthrough assembly to the PCB, wherein the feedthrough wire is connected to the PCB with a pigtail spring connector.

2. The implantable medical device of claim 1, wherein the pigtail spring connector includes a curled shape.

3. The implantable medical device of claim 1, wherein the pigtail spring connector includes a coil shape.

4. The implantable medical device of claim 3, wherein the pigtail spring connector is attached to the PCB at a through-hole trace contact on a surface of the PCB.

5. The implantable medical device of claim 4, wherein the pigtail spring connector includes an axis extending perpendicularly to the PCB surface.

6. The implantable medical device of claim 1, wherein the pigtail spring connector includes a coil shape having an axis extending perpendicular from a surface of the PCB, wherein the coil shape defines a series of diameters that go from larger to smaller as the coil shape extends farther from the PCB surface.

7. The implantable medical device of claim 6, wherein the pigtail spring connector includes a gold-coated wire coil.

8. The implantable medical device of claim 1, wherein the feedthrough wire extends through a central axis of the pigtail spring connector so as to contact an inner surface of a coil shape of the pigtail spring connector.

9. The implantable medical device of claim 8, wherein the feedthrough wire extends through a through-hole in the PCB and goes up though the pigtail spring connector and is tack welded to the pigtail spring connector.

10. The implantable medical device of claim 9, wherein the pigtail spring connector includes a gold-coated wire coil.

11. A method of making a feedthrough wire connection to a PCB for an implantable medical device, the method comprising:
attaching a pigtail spring connector to a PCB; and
connecting a feedthrough wire to the pigtail spring connector.

12. The method of claim 11, wherein the pigtail spring connector includes a coil shape.

13. The method of claim 12, wherein the pigtail spring connector is attached to the PCB at a through-hole trace contact on a surface of the PCB.

14. The method of claim 13, wherein the pigtail spring connector includes an axis extending perpendicularly to the PCB surface, and wherein the coil shape defines a series of diameters that go from larger to smaller as the coil shape extends farther from the PCB surface.

15. The method of claim 14, wherein the pigtail spring connector includes a gold-coated wire coil.

16. The method of claim 11, wherein the feedthrough wire extends through a central axis of the pigtail spring connector so as to contact an inner surface of a coil shape of the pigtail spring connector.

17. The method of claim 16, wherein the feedthrough wire extends through a through-hole in the PCB and goes up though the pigtail spring connector and is tack welded to the pigtail spring connector.

18. A method of attaching a feedthrough wire to a PCB, the method comprising:
extending a feedthrough wire from a feedthrough assembly through a through-hole in a PCB and through a center of a pigtail spring connector; and
tack-welding the feedthrough wire to the pigtail spring connector.

19. The method of claim 18, wherein the pigtail spring connector is attached to the PCB at a trace contact at the through-hole of the PCB, wherein the pigtail spring connector includes an axis extending perpendicularly to a surface of the PCB, and wherein the pigtail spring connector includes a coil shape having an axis extending perpendicular from the surface of the PCB, wherein the coil shape defines a series of diameters that go from larger to smaller as the coil shape extends farther from the PCB surface.

20. The method of claim 18, wherein the feedthrough wire extends through a central axis of the pigtail spring connector so as to contact an inner surface of the pigtail spring connector.
